(19) United States
(12) Patent Application Publication
(10) Pub. No.: US 2008/0201081 A1
(43) Pub. Date: Aug. 21, 2008

(54) Analytical Method and Apparatus

(75) Inventor: Bevan Leslie Reid, New South Wales (AU)

Correspondence Address: KNOBBE MARTENS OLSON & BEAR LLP, 2040 MAIN STREET, FOURTEENTH FLOOR, IRVINE, CA, 92614, US

(73) Assignee: Fermiscan Australia Pty Ltd, Sydney (AU)

(21) Appl. No.: 11/577,655

(22) PCT Filed: Oct. 21, 2005
(86) PCT No.: PCT/AU2005/001623

§ 371 Date: Jan. 22, 2008

(30) Foreign Application Priority Data
Oct. 22, 2004 (AU) 2004906129
Dec. 13, 2004 (AU) 2004907068

(51) Int. Cl. G01N 33/48

(52) U.S. Cl. 702/19

(57) ABSTRACT

A method for analysing function of a biosystem (16) based on analysis of a sample (12) taken from a portion of said biosystem (18); said method comprising exposing said sample to incident energy (10) derived from an energy source (11); receiving radiated energy from said sample consequent to impingement of said incident energy on said sample; passing at least a portion of said radiated energy through a transducer (13) thereby to derive an information signal (15) which characterises an aspect of said sample (12); analysing said information signal to produce biosystem data which can be used to identify said aspect of said sample (12).

The present invention relates to an analytical method and apparatus and, more particularly, to such a method and apparatus suited, although not exclusively, to samples of biological material for the purpose of characterisation of diseases which may be associated directly or indirectly with the biological sample.

### BACKGROUND

Many and varied analytical techniques are known whose aim is to seek to diagnose disease or other defect in a biological sample.

On the other hand techniques for inferring disease or other defect in biological structures which may be directly or indirectly associated with the biological sample but which do not themselves comprise the biological material from which the biological sample has been taken are far less developed at this time if at all.

In addition, a separate problem, is that biological structures, particularly those associated with mammals, are exceedingly complex with the result that often large sample sets and volumes of data need to be obtained and analysed in order to draw any potentially useful conclusions.

The advent of the high speed digital computer has assisted in the processing of large volumes of data but this, in itself, is not enough in most instances to derive analytically useful information direct from biological samples and certainly not from other biological structures which may be associated directly or indirectly with the biological sample.

A patent which describes a method of indirect detection of a pathological state is U.S. Pat. No. 6,718,007 to James. This patent describes use of x-ray diffraction techniques applied in a non-real time mode. There exist also a number of papers describing indirect diagnostic techniques for example associated with the use of saliva as a diagnostic medium—see for example “Salivary Glands and Saliva—Saliva as a Diagnostic Fluid” by Office of Research and Graduate Programs, School of Dentistry, University of Mississippi Medical Centre published early 2002.

Separately there have been a number of patents issued in relation to use of high speed sampling and measurement techniques applied to large numbers of biological samples see for example U.S. Pat. No. 6,780,647 to Fujiwara et al, U.S. Pat. No. 6,794,127 to Lafferty et al, and U.S. Pat. No. 6,778,724 to Wang et al.

Interferometer techniques have been utilised in U.S. Pat. No. 6,330,064. Optical sensing techniques have been utilised in U.S. Pat. No. 6,111,247 to Melendez et al. A long period grating optical device has been used as the basis for a sensing technique in U.S. Pat. No. 6,275,628 to Jones et al.

Finally, the compact disc has been proposed as a platform for various forms of analytic techniques—see for example “Cell Analysis System Based on Compact Disc Technology” by Tibbe et al published in Cytometry 47:173-182 in 2002. See also “Molecular Screening on a Compact Disc” by La Clair et al published in Organic Biomolecular Chemistry 2003 1(18), 3244-3249 in 2003 and finally see also U.S. Pat. No. 6,685,885 to Nolte et al.

All of the above references point to efforts being made in the separate disciplines of high speed automated analysis of samples on the one hand and use of indirect diagnostic techniques on the other but not the two combined. This discussion is not to be taken as an admission that any of the references in this section form part of the common general knowledge or would otherwise be considered as readily combinable with one another.

It is an object of the present invention to address or ameliorate one or more of the abovementioned disadvantages.

### BRIEF DESCRIPTION OF INVENTION

Accordingly there is provided in one broad form of the invention a method for analysing function of a biosystem based on analysis of a sample taken from a portion of said biosystem; said method comprising exposing said sample to incident energy derived from an energy source; receiving radiated energy from said sample consequent to impingement of said incident energy on said sample; passing at least a portion of said radiated energy through a transducer thereby to derive an information signal which characterises an aspect of said sample; analysing said information signal to produce biosystem data which can be used to identify said aspect of said sample.

Preferably, said information signal includes a real component and an imaginary component.

Preferably, said imaginary component is used as a basis for to characterising of said aspect of said sample.

Preferably, said aspect of said sample is a disease or malfunction.

Preferably, said aspect is used to characterise a disease or malfunction of an associated portion of said biosystem.

Preferably, said biosystem is a mammalian system.

Preferably, said mammalian system is the human body.

Preferably, said biosystem includes soil.

Preferably, said biosystem comprises an agricultural system.

Preferably, said step of analysing said information signal includes comparing said biosystem data derived from said sample with biosystem data derived from samples associated with a predetermined aspect of said biosystem.

Preferably, said aspect comprises a disease state.

Preferably, said aspect is characterised at the atomic level.

Preferably, said aspect is characterised with reference to the Fermi surface of atoms comprising said sample.

Preferably, said background reference data is injected into said radiated energy.

Preferably, said sample is scanned repeatedly by said incident energy.

Preferably, said sample is placed on a platform which is rotated relative to said incident energy thereby to cause repeated passes of said sample through said incident energy.

Preferably, said incident energy derives from a laser source.

Preferably, said step of analysing said information signal to produce biosystem data is conducted in real time.

Preferably, said biosystem is a mammalian system.

Preferably, said biosystem includes soil.

Preferably, said biosystem comprises an agriculture system.

Preferably, said mammalian system is the human body.

Preferably, said mammalian system is an animal body.

Preferably, said mammalian system is a horse, dog or cat.

Accordingly there is provided in a further broad form of the invention a device for analyzing biosystem function of a biosystem based on analysis of a sample taken from a portion of said biosystem, said device comprising:
a) a source of energy for exposing said sample to incident energy derived from said source of energy;
b) at least one sensor for receiving radiated energy from said sample consequent to impingement of said incident energy on said sample;
c) a transducer for receiving at least a portion of said radiated energy from said at least one sensor so as to derive an information signal which characterises an aspect of said sample;
d) a processor for receiving said information signal from said at least one sensor wherein said processor analyses said information signal to produce biosystem data which can be used to identify said aspect of said sample.

Preferably, said incident energy includes laser radiation.

Preferably, said incident energy includes space radiation.

Preferably, said radiated energy includes space radiation.

Preferably, said information signal includes a real component and an imaginary component.

Preferably, said imaginary component is used as a basis for characterization of said aspect of said sample.

Preferably, said aspect of said sample is a disease or malfunction.

Preferably, said aspect is used to characterize a disease or malfunction of an associated portion of said biosystem.

Preferably, said biosystem is a mammalian system.

Preferably, said biosystem includes soil.

Preferably, said biosystem comprises an agriculture system.

Preferably, said mammalian system is the human body.

Preferably, said step of analyzing said information signal includes comparing said biosystem data derived from said sample with biosystem data derived from samples associated with a predetermined aspect of said biosystem.

Preferably, said aspect comprises a disease state.

Preferably, said processor processes information pertaining to spaces within and between elements of said stored information.

Preferably, said sample is mounted on an analytical platform wherein said analytical platform includes a support surface for supporting said sample and an analytical layer wherein said analytical layer is connected to said support surface and said analytical layer is positioned below said support surface whereby said analytical layer receives a portion of said radiated energy from said sample so as to perturb at least a portion of said radiated energy wherein said perturbations are subsequently detected by said at least one sensor.

Preferably, said sample includes blood.

Preferably, said sample includes saliva.

Preferably, said sample includes tissue.

Preferably, said sample includes hair.

Preferably, said radiated energy include effects of laser radiation.

Preferably, said analytical platform comprises a CD Rom.

Preferably, said CD Rom is played in a CD Rom player.

Preferably, said at least one sensor includes the sensors located within said CD Rom player.

Preferably, said processor is connected to said CD Rom Player so as to process information received from said CD Rom player.

Preferably, said CD Rom player is located in a container.

Preferably, said container includes temperature and pressure sensing devices so as to accurately trace the ambient pressure and temperature inside said container.

Preferably, said container includes a photodiode for detecting said radiated energy from said CD Rom when said CD Rom is played.

Preferably, playback of said CD Rom is associated with spark discharges inside said container so as to alter the state of said radiated energy.

Preferably, said incident energy and said radiated energy are permitted to pass through a solution of sugar wherein said solution is interposed between said surface of said CD Rom and means within said CD Rom player used to detect said radiated energy.

Preferably, said incident energy and said radiated energy are permitted to pass through a combination of DNA and salt wherein said combination of DNA and salt is interposed between said surface of said CD Rom and means within said CD Rom player used to detect said radiated energy.

Preferably, playing of said CD Rom is performed in a spherical housing.

Preferably, playing of said CD Rom is performed in a cubical housing.

Preferably, playing of said CD Rom is performed in a spherical housing wherein said spherical housing is constructed from aluminum foil or mu metal.

Preferably, playing of said CD Rom is performed in a cubical housing wherein said cubical housing is constructed of aluminum foil.

Preferably, said device comprises placing a lead mass in the immediate vicinity of said CD Rom player and within said container prior to playing said CD Rom on said CD Rom player.

Preferably, said lead mass weighs approximately 10 kg and is at least 3 mm think.

Preferably, playing said CD Rom occurs at night so as to compare the difference in response of said radiated energy between night and day time playing.

Preferably, playing said CD Rom occurs in the day time so as to compare the difference in response of said radiated energy between night and day time playing.

Preferably, playing said CD Rom occurs under differing seasonal conditions so as to compare the difference in response of said radiated energy between differing seasonal conditions.

Preferably, playing of said CD Rom occurs within said container whereby said container is sealed from the external atmosphere so as to enable said container to include an artificial atmosphere of ordinary air.

Preferably, playing of said CD Rom occurs within said container whereby said container is sealed from the external atmosphere so as to enable said container to include an artificial atmosphere of nitrogen.

Preferably, playing of said CD Rom occurs within said container whereby said container is sealed from the external atmosphere so as to enable said container to include an artificial atmosphere which includes argon.

Preferably, the device for analyzing biosystem function of a biosystem based on analysis of a sample taken from a portion of said biosystem substantially as described and illustrated in the body of the specification.

In a further broad form of the invention there is provided a method for analyzing biosystem function of a biosystem based on analysis of a sample taken from a portion of said biosystem; said method comprising the steps:
a) exposing said sample to incident energy derived from a source of energy;
b) using at least one sensor to receive radiated energy from said sample consequent to impingement of said incident energy on said sample;
c) passing at least a portion of said radiated energy through a transducer thereby to derive an information signal which characterizes an aspect of said sample;
d) using a processor to analyze said information signal to produce biosystem data which can be used to identify said aspect of said sample.

Preferably, said energy includes heat energy.

Preferably, said energy includes sound energy.

Preferably, said energy includes electromagnetic energy.

Preferably, said incident energy includes space radiation.

Preferably, said radiated energy includes space radiation.

Preferably, said information signal includes a real component and an imaginary component.

Preferably, said imaginary component is used as a basis for characterization of said aspect of said sample.

Preferably, said aspect of said sample is a disease or malfunction.

Preferably, said aspect is used to characterize a disease or malfunction of an associated portion of said biosystem.

Preferably, wherein said biosystem is a mammalian system.

Preferably, said mammalian system is the human body.

Preferably, said step of using a processor to analyze said information signal includes comparing said biosystem data derived from said sample with biosystem data derived from samples associated with a predetermined aspect of said biosystem.

Preferably, said aspect comprises a disease state.

Preferably, said processor processes information pertaining to spaces within and between elements of said stored information.

Preferably, said sample is mounted on an analytical platform wherein said analytical platform includes a support surface for supporting said sample and an analytical layer wherein said analytical layer is connected to said support surface and said analytical layer is positioned below said support surface whereby said analytical layer receives a portion of said radiated energy from said sample so as to perturb at least a portion of said radiated energy wherein said perturbations are subsequently detected by said at least one sensor.

Preferably, said sample includes blood.

Preferably, said sample includes saliva.

Preferably, said sample includes tissue.

Preferably, said sample includes hair.

Preferably, said radiated energy includes effects of laser radiation.

Preferably, said analytical platform includes a CD Rom.

Preferably, said CD Ram is played in a CD Rom player.

Preferably, said at least one sensor includes the sensors located within said CD Rom player.

Preferably, said processor is connected to said CD Rom Player so as to process information received from said CD Rom player.

Preferably, said CD Rom player is located in a container.

Preferably, said container includes temperature and pressure sensing devices so as to accurately trace the ambient pressure and temperature inside said container.

Preferably, said container includes a photodiode for detecting said radiated energy from said CD Rom when said CD Rom is played.

Preferably, playback of said CD Rom is associated with spark discharges inside said container so as to alter the state of said radiated energy.

Preferably, said incident energy and said radiated energy is permitted to pass through a solution of sugar wherein said solution is interposed between said surface of said CD Rom and means within said CD Rom player used to detect said radiated energy

Preferably, said incident energy and said radiated energy is permitted to pass through a combination of DNA and salt wherein said combination of DNA and salt is interposed between said surface of said CD Rom and means within said CD Rom player used to detect said radiated energy.

Preferably, playing of said CD Rom is performed in a spherical housing.

Preferably, playing of said CD Rom is performed in a cubical housing.

Preferably, playing of said CD Rom is performed in a spherical housing wherein said spherical housing is constructed from aluminum foil or mu metal.

Preferably, playing of said CD Rom is performed in a cubical housing wherein said cubical housing is constructed of aluminum foil.

Preferably, said method comprises placing a lead mass in the immediate vicinity of said CD Rom player and within said container prior to playing said CD Rom on said CD Rom player.

Preferably, said lead mass weighs approximately 10 kg and is at least 3 mm think.

Preferably, playing said CD Rom occurs at night time so as to compare the difference in response of said radiated energy between night and day time playing.

Preferably, playing said CD Rom occurs in the day time so as to compare the difference in response of said radiated energy between night and day time playing.

Preferably, playing said CD Rom occurs under differing seasonal conditions so as to compare the difference in response of said radiated energy between differing seasonal conditions.

Preferably, playing of said CD Rom occurs within said container whereby said container is sealed from the external atmosphere so as to enable said container to include an artificial atmosphere of ordinary air.

Preferably, playing of said CD Rom occurs within said container whereby said container is sealed from the external atmosphere so as to enable said container to include an artificial atmosphere of nitrogen.

Preferably, playing of said CD Rom occurs within said container whereby said container is sealed from the external atmosphere so as to enable said container to include an artificial atmosphere which includes argon.

### BRIEF DESCRIPTION OF DRAWINGS

Embodiments of the present invention will now be described with reference to the accompanying drawings wherein:

FIG. 1 is a block diagram of a diagnostic system and associated apparatus in accordance with a first preferred embodiment of the present invention;

FIG. 2 is a further block diagram of a particular implementation of the apparatus of FIG. 1;

FIG. 3 is a diagram of a method of analysis of the sample of FIGS. 1 and 2 at the atomic level.

FIG. 4 is a schematic diagram of the system of FIG. 1 implemented utilising CD or DVD technology.

FIGS. 5-8 support the description according to a second preferred embodiment.

### DETAILED DESCRIPTION OF PREFERRED EMBODIMENTS

### First Embodiment

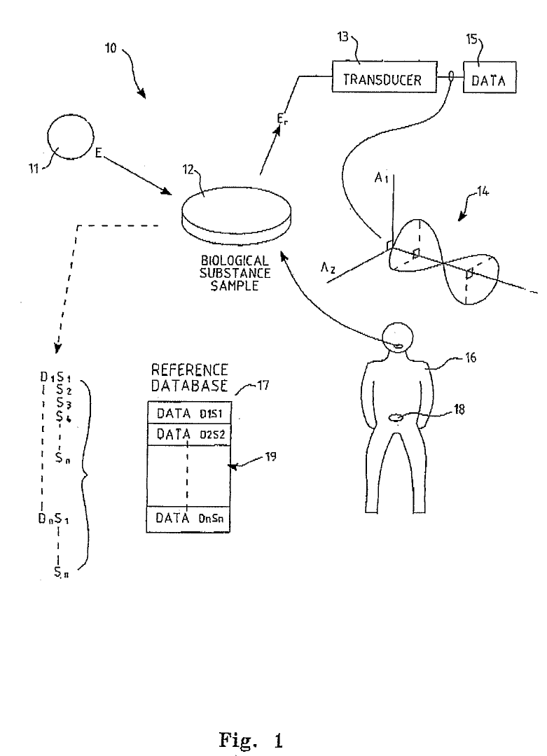

With reference to FIGS. 1 to 4 there is illustrated a diagnostic system 10 which includes one or more of the following features:
1. diagnosis of characteristics of structures associated directly or indirectly with the sample—not just the structure from which the sample was taken
2. the analytic platform or layer located below the support layer for the sample as for example used in the CD implementation to provide a background data source for reference purposes
3. analysis with reference to Fermi layer concepts and derivation of data at the atomic level from the sample and reliance on that data to infer clinically useful information.

With reference to FIG. 1 there is illustrated a diagnostic system 10 according to a first preferred embodiment of the present invention. The primary components of the system 10 are an energy source 11 which is arranged to cause energy Ei to impinge on or otherwise irradiate a biological substance sample 12. Consequent to impingement of incident energy Ei on biological substance sample 12 radiated energy Er is radiated from sample 12. A transducer 13 is adapted to receive at least a portion of the radiated energy Er and to convert that portion of energy into an information signal 14 which contains information components which characterise an aspect of sample 12. The information signal may either be stored directly as sample data 15 either directly or following an information processing step as processed data which contains information which characterises an aspect of sample 12. Typically the data 15 will be stored as digital data.

The biological substance sample 12 is derived or taken from a biosystem 16. In this particular instance the biosystem 16 is that of the human body and the sample 12 comprises a sample of saliva (or serum or hair).

Diagnostic system 10 also includes a reference database 17 which, in this instance, stores a series of sample data D1S1 . . . D1Sn . . . DnS1 . . . DnSn derived from respective samples S1 . . . Sn for samples taken from biosystems exhibiting symptoms of respective diseases or malfunctions D1 . . . Dn.

The disease or malfunction in question may be associated with and exhibited by the respective samples themselves or the disease or malfunction may be associated with some other portion of biosystem 16. In this latter scenario the sample 12 taken from biosystem 16 is selected to be of a sample type which will include information indicative of the malfunction or disease of the associated portion 18 of biosystem 16.

By way of non-limiting example where the biosystem 16 is that of the human body and the sample 12 is saliva (or serum or hair) the associated portion 18 can be, for example, the liver with the saliva sample 12 including information pertaining to malfunction or disease of the liver.

Diagnosis of a disease or malfunction of a portion of biosystem 16 is performed by comparison of sample data 15 with the reference data samples 19 comprising, in this instance, samples D1 S1 through to Dn Sn in reference database 17. Appropriate statistical analysis can allow inference of malfunction or disease states of biosystem 16 to a predetermined level of certainty. The levels of certainty can be improved by increasing the number of reference data samples 19 in reference database 17.

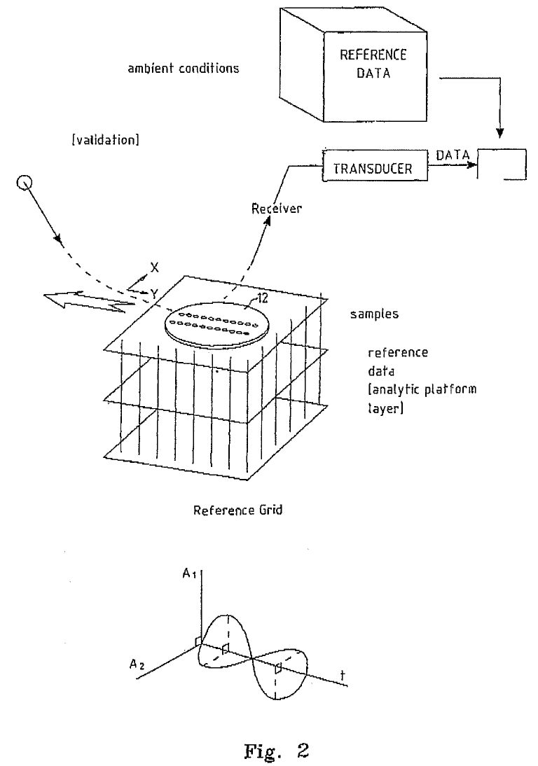

FIG. 2 illustrates in greater detail a referencing system relating to both three dimensional space and data space which can be utilised in one particular embodiment of the present invention.

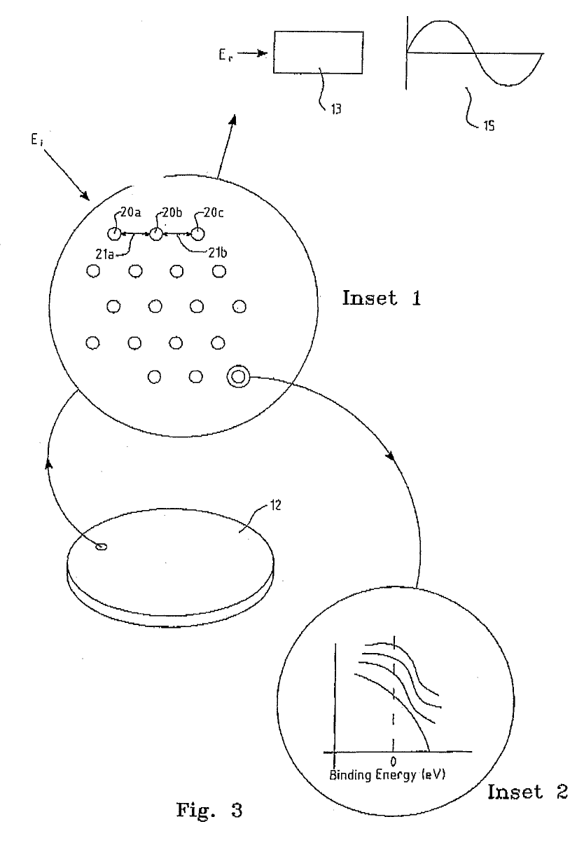

FIG. 3 illustrates diagrammatically and conceptually one particular approach to deriving information characterising an aspect of sample 12. The inset in FIG. 3 can be viewed as a high level magnification of a portion of sample 12, magnified to the point of showing atoms 20a, 20b, 20c making up that portion of the sample 12 and more particularly the interatomic spacing 21a, 21b . . . between atoms 20a, 20b.

Depending on the makeup of sample 12 its atomic structure may be regular or irregular and typically will, in fact, vary in a highly complex way.

In one preferred embodiment of the present invention it is sought to derive an information signal 15 characterising an aspect of sample 12 with reference to analysis at the atomic level of sample 12. In one particular form, as will be discussed in greater detail below, this can be done with reference to fermi-levels and the fermi-surface—concepts utilised in solid-state physics, particularly in the case of metals.

With further reference to inset 1 and inset 2 FIG. 3 it is possible to characterise the atomic structure of atoms 20 with reference to Fermi surfaces. The literature defines a Fermi surface as the locus of points in momentum space with zero excitation energy. The topology of atom 20 can be displayed graphically resulting from a mapping of the locus of zero excitation energy points as shown by the dotted line in inset 2 of FIG. 3. The Fermi surfaces of atoms 20 can form the basis for characterisation of an aspect of sample 12. With further reference to FIG. 2 this characterisation may be a function of position of the atoms within sample 12. In the alternative an averaging technique can be used to obtain a bulk characterisation of sample 12 with reference to the Fermi surfaces of the atoms making up sample 12. It will be observed that this technique seeks to characterise sample 12 by measurements at the atomic level, which is to say measurements of the order of 10⁻¹⁵ (units of this dimension are termed Fermi units).

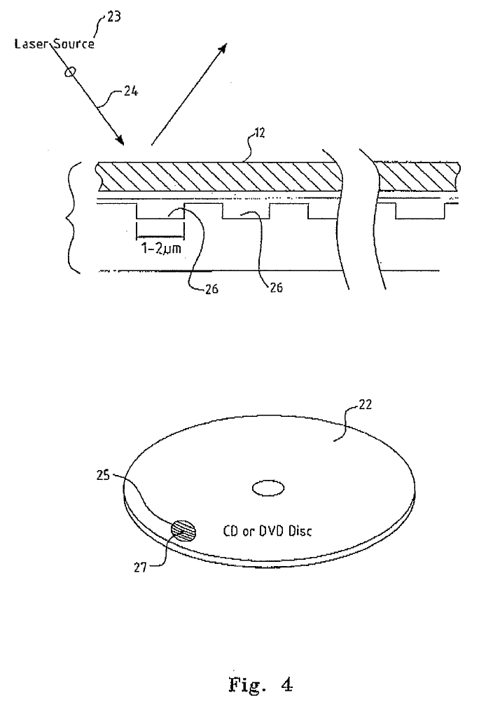

With reference to FIG. 4 a characterisation of sample 12 at the bulk level but still with reference to a reference grid can be performed utilising CD or DVD disc 22. In this instance a laser source 23 directs a laser beam 24 onto sample 12 which overlays at least some tracks 25 of disc 22. As best seen in the inset of FIG. 4 the tracks include pits 26 which are typically of the order of 1-2 microns in length and width and may be of a depth of the order of 1 micron or less.

The beam 24 can be located by a control system (not shown) so as to have its point of focus 27 on disc 22 ascertained to better than one micro metre thereby allowing features of sample 12 to be resolved to the order of 1-2 micro metres.

### Second Embodiment

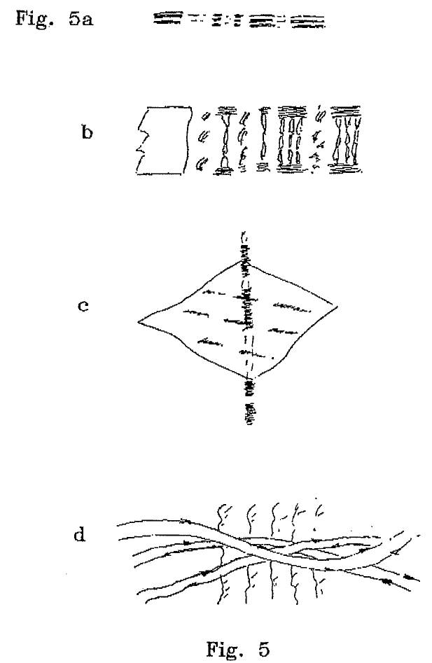

With reference to FIG. 5 onwards some of the core concepts which may be applied to the analysis of the sample 12 at or characteristic of the atomic level are discussed more comprehensively and in non-limiting fashion below.

### Introduction; A Brief Historical Survey

Some two millennia after the Greeks came to break up the continuum of matter when Democritus segregated the ultimate in indivisibility (the a-tom) the other great continuum, energy, has suffered the same fate. At the origins of the industrial revolution engineers such as Carnot derived mathematics to cope with the cycles produced in the engines with the effect of subdividing the energy form, heat from its continuum, his so called “chute de calorique”. Some decades later the mathematician Clausius segregated local space entities according to their volume (and thus their pressure) in dividing the very abstract subdivision of entropy.

Another mathematician, Clerk Maxwell, collected the quantities of electricity and magnetism proposed at the time (ca 1860) with four equations soon to be recognised as constituting a distribution of energy as waves. Solutions of these equations produced waves whose length came to afford a distribution which, pleasingly, could accommodate phenomena such as heat and light to which later (last quarter of the century under notice) was added radio wavelengths. Hertz and other investigators at the time, discovered that light beams of an appropriate magnitude aimed at metals could dislodge electrons, the so-called photoelectric effect. The continuity of energy was thus yielding to wave ideas and the waves were distinguished clearly by their length. Behaviour based on length was soon to follow.

If we dwell on one part of Maxwell's distribution, by now termed a wave spectrum (light), the discretization idea preceded the electromagnetic by several decades when the physician, Young, noted that a slit in a light shutter or blind, admitted the light, not as a continuous slot corresponding to the slit, but as a series of dark lines whose pattern became the more complex in the presence of a second adjacent slit admitting the light source. The pattern was to be expected of the interaction of water waves in a pond so that the wave theory of light was acceptable, ready for accommodation into Maxwell's formalism.

When the same agent, light, was found to dislodge electrons under defined conditions from the initial target as described, the concept of a ballistic, termed a particle, somehow inserted into the wave, became current at about the turn of the last century. The specificity of the electron dislodgement spawned the idea, that an older idea from mechanics, that energy could be equally partitioned amongst the waves and their respective lengths, was a resounding assurance from the classics, that energy partitioning possibly involving equality, was quite valid.

It was at this point that perhaps the greatest advance for an unsuspected discretisation of energy associated with the proposal of its equal value amongst wavelengths, was discovered, not in any fortuitous way, but as a result of several months of hard mentation (“amongst the hardest work of my life”) on the part of the thermodynamicist—theoretical physicist, Max Planck in Germany. By considering the energy curves produced by radiant heat as applied to varying wavelengths, Planck noted it was not symmetrical in the Gaussian way to be expected from the classical theories of equipartition, but that, as the wavelength got shorter (or the frequency increased in more usual terms) as to approach the ultraviolet wavelength, the heat curve became asymmetrical in the sense that relatively less heat was required to sustain this part of the wavelength curve. In other words, at those higher frequencies, the waves seemed to be interacting in a fashion not occurring at lower frequencies. Planck brought his equations for the phenomenon from proportionality to equality in the usual way, by the use of a constant which he termed h. It was a feature of the formalism, that the wave bore the constant h at one whole wave length (or revolution) which he termed thereby, a quantum.

Although the magnitude of h is now well known in angular momentum terms, its function in considering the energy of higher frequency waves is presently unknown but some of its qualities bear directly on a division in the discretization of energy of direct value to the argument advanced here. We refer to a bereavement of quantum energy from the energy form, heat.

### The Bereavement of Heat

A true paradox might have been forecast from the mathematics of the photoelectric effect that its term was h x frequency i.e. it bore no evidence of the usual kT (where k is Boltzamann's Constant and T is temperature at the Kelvin scale and kT had been known as an energy packet moving molecules,) but stood alone unqualified. It was twelve years after his discovery of the constant, that Planck's formalism was able to dispense with T meaning that the agent dislodging the electrons in the photoelectric effect, together with that condensing waves at higher frequency, was not using heat for these effects, which sent Planck into a remorse on the apparent meaninglessness of all that he had discovered after all that work: some factor that was a complete paradox a complete abruption, especially from his experience as a trained thermodynamicist.

In the pace of events in this early part of last century, it was widely grasped but somewhat indifferently that Einstein (1905) had noted this independence of heat in his own treatment of the formalism, and averred that it could only occur were this basic quantal energy to persist at 0° K as ½ Planck's constant X frequency as a negative and half as a positive value for the same term. So was born the concept of an energy at Zero Kelvin, in other words, an energy unrelated to temperature, veritably an energy of pure space itself, all this a century ago.

This should not have been surprising in that there was considerable energy in the photo-electric term sufficient for it to be dubbed a work function, again an idea with a seniority of over a century. Even in the half or more century to follow, physicists such as De Witt clung to the classical heat component argument (1) (at least at the level of kT) and we are reluctant to pass up this discussion with its elements of disbelief amongst highly informed opinion, without a brief excursion into the world of epistemology, the better to sustain an important theme in this presentation: that it is perfectly bona fide to view pure pristine space as a source of one compartment of energy in its own right. General relativity does not always underline this proposal, so that restatement may be warranted.

### An Epistemological Glimpse

Space has no measurable feature available to modern man and his specialist (physicist) adjudicators. The prejudice shown by De Witt is an example of those very informed physicist minds who have a distaste of dabbling in the non-matter realms of pure space. Indeed they tend to regard those discussions as metaphysical. The resolve to abolish nought (or infinity) from the equations by the process of renomalisation has a seal of approval stretching to the Committee responsible for the Nobel prize in physics on more than one occasion. So entrenched is the attitude, that in seeking an explanation, it were better to avoid the superficiality or the trivial of prejudice, and seek a deeper meaning for the impasse.

Western science is unashamedly proud of its origins in Greco-Roman logic. Over those eons, the concepts of Plato, (there was an object and there was its form) was summarily dismissed by Aristotle and the several scholars who followed him, over the centuries. There were islands of support for the concept of form (we might now say geometry) within matter over the centuries but, until quantum theory and the zeropoint energy, there was little real support for alternatives to the thing (matter) as central in causation.

How is this so, we ask? And all this in the knowledge that much formalism contains i in its terms in modern equations.

The query has raised the attention of one or two mathematician writers recently, who in their enquiry, trace the origin of the use of nought, to a bookmaker's clerk in Padua about 1480. He advised his master that he would pursue odds calculations through the imaginary world by use of the term i. Scholars see this as a source point for the western world's interest in the term which we note at circa five hundred years ago. If we assign about thirty years to a human generation, in other words about fifteen generations, this seems, in genetic terms, far too short for the establishment of a critical mass for the concept as a whole. Nought has to persist outside the pale for a while yet!

We deem this excursion as worthy, if space as an entity in physics and chemistry is to be promoted further. It has worthy detractors who needs be accommodated before we abandon the whole concept.

Progress in the Space Concept following Planck and Einstein

Pondering the meaning of the clearly strange features of the energy of the quantum field, Heisenberg, using as basis a matrix algebra, suggested that the precision in terms used in numerical algebraic methods did not allow of customary statement more especially of position in space and velocity, in dealing with quantum theory. He was convinced that the usual precision was here unknowable because of their uncertainty in his formalism.

A few years later the British engineer Dirac took right sided equation terms to the left side in deriving new quantal field equations, a sort of inversion, and came up with the idea that simple particles such as the electron (which because of its mass could be considered real), was the real world counterpart of a sea of unreal, unobservable counterparts in the imaginary world which, in antithetical fashion he termed positrons. The demonstration of elements with just such properties by physicists just a couple of years later, did little to still the disquiet of the need to take the imaginary world of space seriously. To avoid its use, the word particle was substituted in the new discipline.

About the same time, Casimir (1) at the Philips Einthoven laboratories, showed that two metal plates in a vacuum could not be held apart without the use of a counteracting force, a force measurable these days in tiny fractions of a Newton (2).

Also in a vacuum, this time in the presence of microwave fields, Lamb and Retherford (3) in the United States were able to split the energy holding the single orbiting electron of hydrogen into two values concluding that the restraining force in the absence of their microwaves in vacuo could only have come from Einstein's vacuum energy field.

From the Dirac work of the thirties onwards then, a whole discipline emerged of the properties of the imaginary world termed quantum electro-dynamics. It was rapidly used, amongst other outcomes, to comprehend the presence of noise in oscillators projected into their output as an obligatory component, noise which persisted in the vacuum state.

In this synoptic historical overview, with its penchant for suggesting pure space as a valid compartment in the discretization of energy forms over the past two centuries, it is supportive to refer to another temperature-free energy form of wide use in thermodynamics known as free energy. This mathematically derived entity was introduced separately in the nineteenth century by Gibbes in the United States and Helmholz in Germany, from considerations of equilibrium states in thermodynamics where the temperature played no part in the energy behaviour because it was kept constant throughout the process. The matter is raised not because temperature is involved in the equations (to reach the equilibrium state) but because Helmholz saw that a single term, not involving temperature, was sufficient to account for the photoelectric effect. Helmholz was want to invoke the interaction of vortices in the establishment of temperature-free states and we will involve these energy structures later in the same way: it illustrates the behaviour of a pure space force when it comes to mimic-real world events such as the progress of vertical streets in water in the discipline of hydrodynamics.

### The Response of Chemicals to Vicinal Space Element Behaviour

There has grown up over the centuries, often through folklore, unusual or esoteric behaviour in living systems, which have no explanations in classical approaches. We choose here to involve the ample representation of chemicals in the living system and suggest that it is to this component of matter, that one should look to examine whether space with force, conceptually if not experimentally extant in physics for over a century, plays any part in chemical behaviour, and if so how such space and its elements may motivate the chemical. In our short dalliance with epistemology, it would be apparent that any such examination would need be rather novel or abrupt in the introduction of tenets that it might embrace, given the contemporary grasp of physics and chemistry. In the world of cause and effect, if matter (such as the chemicals) is clearly dominant, surely there is nothing else. Even more perniciously expressed, non local effects or cosmic events, lunar, solar, planetary, so obvious in biosystem behaviour, would await explanations merely from more exhaustive analysis and experimentation using extant precepts.

With this ‘something else’ precept in mind, it is not difficult to assemble observations and experiment using relatively inexpensive procedures to show that space itself does have important effects on chemical behaviour more especially using the parameter of non-locality. Some agent such as a space component could be acting over distances way beyond atomic and molecular dimensions more particularly by its association with nearest neighbour space particles, in domino or percolative fashion.

If we start with an analysis of present precepts held by a variety of workers in fields of mathematics, theoretical physics and physical chemistry, it is possible to synopsise, at least in outline, a notion of a structure and function for space adhering closely to received extant knowledge. It is convenient to divide this synopsis into the local and the non-local, respectively within the atom and more distantly in its neighborhood and thence to the Cosmos.

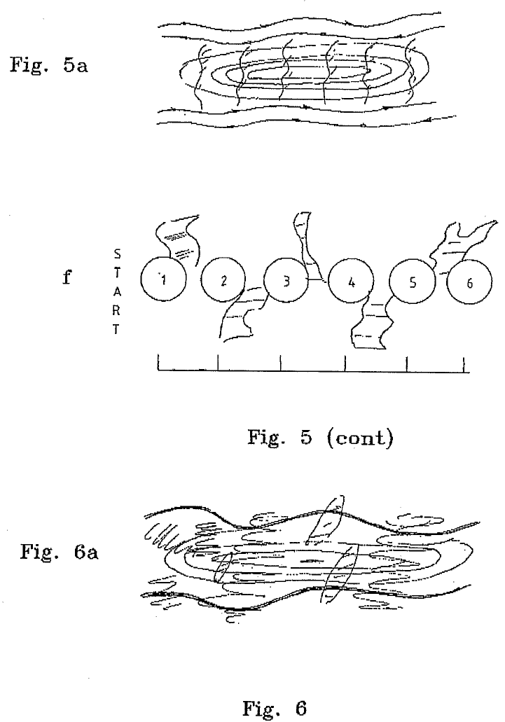

Imaginary energy for the physical chemist, occurs in linear and planar dispositions, the former being composed of a bi-directional pair, the latter set perpendicular to the former and displayed as a series of lines in plane form (FIG. 15). The picture is not too different from the Euclidean infinite wave of mathematics along the course of which occur planar wavelets. Although the co-linear fraction travels between atoms and molecules linking them, the orthogonal fraction nests within the atom or molecule, varyingly interposed between the energy elements forming the nucleus and the orbiting electrons, more especially the valency orbits(5) (FIG. 5e, FIG. 6a). It is possible, but not researched, that the collinear fraction joins its collinear fellows within the dynamism of the living organism, to form conjoined bundles in the nature of the meridian of Traditional Chinese Medicine, and in that case, its elements have an interchange with the external environment via “holes” in the integument. This means that the valency electron behaviour could ultimately respond to more distant environmental space signals as if responsive to some primitive nervous system.

The precise elemental arrangements at the junction, collinear—orthogonal, are not spelled out in the literature, but if the collinear stream splayed out amongst the orthogonals, to be recollected on the exit side of the interaction, there would then be a dimensional transition 1D of the former to 2D of the latter in space flow at each atom and molecule. This circumstance may be of pivotal value in subsequent mathematical treatment of these flows as we later discuss.

Another scholar in the United States treats two divisions of space element structure in his quest for the ultimate nature of the photon. His formalism provides for a Euclidean infinite wave proceeding either way in one dimension, as a series of integers, 1, 2, 3 . . . n, and they are associated with a subdivision of a space revolution into four quarter-wave entities each of which is planar and (except for the third quartile) allowed to fold or pucker the plane as does a flag (FIG. 5f). The formalism determines that this folding is under the control of Planck's constant h (an angular momentum entity as discussed) and its sequential behaviour, folding—unfolding, is therefore uncertain or unknowable. He illustrates this behaviour as a series of vortices of varying structure from spindle—(or lozenge-) shaped to its reversed complement, narrow in the middle and flared at either end. Assigned to the quartile and showing a flux in vorticeal shape transition of uncertain format, we draw the conclusion that the two dimensional component is in considerable flux which the noted French physicist Prince Louis de Broghie saw as a frenzy. Wunderman's insight (5) had a further pivotal point. He was wanted to start the Euclidean count at a point say point 1. This concept of “starts” is not all that uncommon in descriptions of space behaviour especially where the student wishes to delineate these space elements from all space in the nature of what could be called a proprietary fluctuation. He patterned the first 8-10 integers differently (5) to those constituting the chain up to n, in that the associated wave cursor describing the sine wave in each pair of half waves was accompanied discretely by time. It was thus time symmetric as opposed to linear placed integers above this point where time was not followed cursorily but rather asymmetrically. This is a point not often dissected by mathematicians even though they are aware that the different times symmetric-asymmetric may turn out to be important. For instance it means that the uncertainty for that whole wave revolution determined by its' orthogonal part is not cancelled and the start of the fluctuation is truly uncertain and thus non linear.

We see then in the dynamic behaviour of the space components within the atom in Wunderman's viewpoint, a great volume variation dependent on the uncertain vorticeal patterns related to their Planck constant angular momentum. This volume variation sets up a recognisable pressure variation because, in its intra atomic locale it is played out in the surrounding space often termed the atomic lattice as a boundary defined as the inner (or first) Brouillon Zone within which space forms part of the zero point energy as already described. The alternation so produced, alters the sign on the pressure of the collected vortices and it was one of these sign alternations which accompanied the radiation as predicted by Einstein in 1905. To invoke pressure requires some discussion at this point, because we are still in the imaginary world. We therefore resort to the mathematician's picture of pressure as a matrix of real and unreal moieties, each of which has its gradient. This still leaves open the origin of the gradient at which point we introduce the non-local aspect of cosmological space, persisting now with the pure synoptic mode of description.

The space energy flow of the heavenly body follows two patterns; the convergent and the divergent divided respectively toward the centre of the body and, once beyond its boundary, to a divergent path collected in a stream toward its journey to the next body. It is in the nature of space element arrangement and interaction, that these patterns come to saturate the matter or the body concerned with like dispositions to the cosmic scale, each counterpart being reflected on a microscopic scale within the components of that matter, that is the atoms and molecules. The space elements external to the interatomic frenzy then exert a counter force which takes the form of a pressure alternation represented in the older German literature as atomic “Zitterbewegung” or simply jitter. Some use the term “breathing” for the phenomenon in large molecules, nucleotides, proteins and the like.

It would not be surprising that the possible significance of a considerable state of flux had escaped the attention of most physicists over the years and we have recalled a couple of points in the historical past due to Casimir and Lamb and Retherford where these pressure variations came to light in the vacuum state.

### The Fermi Surface of the Atom

It is not further surprising then, that a forum for these interactions at the junction of dimensionally different flows, collinear and orthogonal, emerged and, in the way physicists have of celebrating their pioneers, it became known as the Fermi layer, We should quite early in the discussion, point out the convenience we attach to a descriptor for the forum given the conceptual requirement that arises in any situation where space energy is to be featured. In that vein, we assign the term Fermi in the understanding that its discovery and associated voluminous research work was made on its status in metals. We suppose gratuitously, that some homologue will eventually be discerned in the case of non-metal atoms.

As lowly massive bodies, electrons are pelagic to many of the flows we have discussed including the interaction of orbiting electrons and those caught up in nuclear-orbital interchanges. Pockets of electron dense and electron poor states occur at the Fermi Surface, the latter termed holes so that the overall pattern is one of bands, evidently in a ceaseless state of perturbation. There is a further rider to this state, in that, as the orthogonal condition moves away from 90° in either direction, so does the Fermi Surface re-establish in a stable state as could be reasoned from energy conservation principles. Such fractional dimensionality changes are most important to this discussion.

Within the flux at this surface then, we have identified a pressure push-pull, as vorticeal motion adapts elastically to a reaction from the vacuum energy, but there is a more subtle movement as a result of the intra-atomic vorticeal collections. In their interaction with phonons, they accelerate to a second order that is within a plane towards or away from each other. This promotes qualitatively different but important reaction from ambient electric and magnetic fields, which, according to the formalism of the Maxwell equations leads to an envelopment of the vorticeal collections as in an electromagnetic wave (FIG. 5a). The second order acceleration sometimes known as the ‘moving mirror radiation’ was proposed separately by Davies and by Unruh and the phenomenon now bears their names. It has possible significance with its basis in reflection of space elements wherein under the appropriate acceleration, radiation including that of light will follow (see later).

### Dimensional Aspects of the Fermi-Surface

In discussion of space flows in the atom it was pointed out that the flow entering the electron-atomic nucleus system was disposed orthogonally to the colinear flow and further that this orthogonality was disposed as a two dimensional plane. For reasons beyond these present discussions it could be that the shape (or topological) conditions on this plane as a result of the energy frenzy occurring here is a surface contour irregularity in point of fact. Equally factual is that this irregularity will vary each time use is made of the Fermi-Surface to fashion that atom. This means that although the chemical will be the same, say carbon, hydrogen, the space structure that it was made in or on will vary in its planarity. The irregularity of planarity referred to is a variant of an integer say 1,2. It is a fraction and a fractional dimension is known as a fractal.

This means that if we are seeking or describing the fate of a chemical in a system whose atoms are constantly moving, it will be essential to define the fractal upon which it is functioning. As part of a plane, each fractal amounts to a fold on that plane. The description of the chemical will be inadequate in a complex functioning system such as a biosystem until its fold of operation is nominated.

### An Ontogeny of the Radiation Field

This brings up the topic of radiation from a source ultimately related to the interaction of the two flows, collinear and orthogonal more widely understood in dielectric theory as the near field of radiation. Engineers usually start from the origin of the near field as it progresses through the intermediate field to the far field of classical radiative phenomena where the envelopment by electric and magnetic waves maybe later joined by waves from the infrared part of the spectrum in the form of heat.

It is convenient for subsequent discussion, to outline the complexity of vorticeal interaction, which may include a second order accelerative phase just prior to envelopment. The more fastidious the parameters for envelopment toward radiation, the more difficult it will be for very precise conditions to occur such that a division at the Fermi Surface locus between vorticeal and radiational elements can be foreseen. It is this very division which we wish to highlight because any imbalance in either part, the push pull of the unenveloped for the one part (FIG. 6b) or the classical fields of electricity and magnetism toward the envelope for the other (FIG. 5b) will result in an imbalance of equilibrium allowing build up of a surfeit of the interactants on either side. If we concern ourselves with the unenveloped side, an equilibrium state could be installed which tends toward an over push or an over pull (FIG. 6c). Specifically, in the instance of the biosystem, this can supply the adjacent chemicals with an enhanced activity level with which, in this theory, is associated in extreme cases an enhanced or uncontrollable growth such as could be expected in neoplasia.

The division referred to has a further important characteristic, in that the vorticeal interactions as pure space elements are not observable. The observability enters only at the second division anlage, the near field-intermediate field in dielectric terms, where the radiation is now observable and can be measured by a variety of instruments, ammeters, thermometers, photoelectric screens and so forth (FIGS. 5a and b). From the specific investigative query of this essay, it emerges from the discussion that a sought after parameter in the origin of neoplasia, the vorticeal imbalance in equilibrium, is limited to the unobserved world, a world described by Wunderman(5) where nothing is known and nothing can be known because of its uncertainty and unreal status (FIG. 5f). It is an entirely non-linear world in marked contrast to the enveloped Maxwell wave, which is predictably linear or quantifiable by observation in its status.

Behaviour in the Spectrum Below Electromagnetic Wavelengths—Linear versus Transverse Waves

To this point the discussion has centred on the relation between the space elements and matter in the form of atoms and molecules so that, not surprisingly, the wavelength of the waves involved has been in the angstrom and nanometre (sometimes termed the optic or visible) range of the spectrum. This range is clearly ideally suited to atomic and molecular magnitudes.

If the discussion is to continue its focus on the biosystem, then it is equally clear that longer, sometimes much longer, wavelengths measurable from micrometers to centimetres to meters to kilometres in length have to be considered. For instance the nanometre band is clearly of direct importance to the functioning chemicals as we indicate but there can be no denial that 1-30 cps waves are essential for brain function and here the wavelength is enormous.

As this length increases from the long radio waves of electromagnetism on toward ultrasonic and sonic frequencies, as is well known, the wave changes from the transverse of the E and B fields of electromagnetism envelopes to a linear wave reliant on its properties by the linearity of elements which it alternatively compresses and decompresses in its flight not unlike on a macroscopic scale the push pull we have described for imaginary waves of interatomic space (FIG. 1c). Thus the linear wave of long wavelengths, say those of ultrasonic and lower modes, may require an altered descriptional stance as compared to that we have used for the transverse waves of electromagnetism.

It is possible to picture sound waves as pressure lines radiating from a point using imaginary radi. It is then possible to view the intersection of circles crossing these radi and at the same time, those circles linking coherent points of high and low pressure in the radiating sound or other long wavelength wave with imaginary planar surfaces cutting these radii (FIG. 1c). They will look ever the more planar the more distant the intersection from the source. These orthogonal planes then become two dimensional information about the pressure status along the line of the wave itself. The essence of this excursion into sound wave structure is to establish that the linear wave can, in this view, become analogous to the transverse wave in possessing an imaginary intersecting one and two dimensional structure. In the case of the origin of the transverse wave, this situation was described in the intratomic site as the interaction of space waves. It would be pleasing if the description could follow analogously in the case of the sound wave such that they too had an imaginary counterpart in two dimensions, the two-dimensional intersecting the one.

Just such a situation has been emerging over the past century in mathematics with the entry of wavelet theory. Here the linear wave is considered as imaginary but its orthogonal off shoot, the wavelet, is usually considered as real. An unreal furnishing of the linear wave with an imaginary orthogonal off shoot would therefore be welcome in our pains to analogise the dimensional mix of the linear wave of sound with the transverse of electromagnetic radiation (FIG. 1c). Just such an event is in prospect from the mathematicians who recently have come to predict orthogonal imaginary planes erected on the linear sound wave eventually to be made real as wavelets. These imaginary planar orthogonals, they provisionally term ridgelets. If we could assign pressure variations in the unreal part of the pressure matrix then the analogy would be completed. In the production of sound and its intra- and supersonic relatives, the analogy would then predict extensive perturbation of the interactive site between the two space dimensions of one and two, thus between the linear wave and its orthogonal ridgelet(6). We theorise that the interridgelet length along the linear parent could be constructed in the optic or nanometre range. This interaction could be interrogated at the optic wavelength on the point that some of the perturbation would be pitched at their optic wavelength as happens for instance in the light flashes of sonoluminescence or in miniscule magnitude in Cerenkor radiation from electromagnetic sources. This posit is tantamount to the suggestion that linear waves at their interaction with matter can suffer an orders of magnitude reduction in wavelength (often termed an attenuation) and there is considerable evidence for the phenomenon in the physics literature. [See for example magnetoacoustic attenuation]. Earlier architects of phonon structure near seventy years ago termed these states respectively auditory and optic phonons.

The Transverse bonded conceptually with the Linear: Scaling

The reason for this detailed pursuit of a possible analogy in the two wave groups transverse and linear is that a need to manipulate imaginary waves at least in mathematical terms is desirable, almost obligatory, given the importance we are attaching to the behaviour of linear in addition to optic or transverse waves. In fact we are attempting the establishment of an important commonality in the two wavelength groups as regard their imaginary or space component: both admit of a partition in dimensionality between one and two in wave interactive behaviour. It will also permit of uniformity in procedures for a method to be described to permit a cursory review of all wavelengths and their harmonics from the optic to the e.l.f. in the important manoeuvre of scaling.

### Review of the Discretization of the Energy Concept

This review was undertaken as prelude to a comprehension as to just how the dynamic behaviour of space, replete with presently immeasurable forces, some of them posited as central to the function of the living matter system, can themselves be measured in a way not possible in the long history of math and physics preceding the appearance of the compact disc, with its implicit Boolean logic.

This view has developed the following points:
1. It is possible to assign a structure and function to the elements of space, parameters which at the same time do not alter the indeterminate value of the structure of the elements themselves nor of the way they are obliged to interact by their possession of a small added angular momentum fragment known as Planck's constant.
2. These elements are omnipresent in the universe where they demonstrate omnipotence, features which penetrate as well, the considerable space volumes in atoms and molecules.
3. The elements are conveniently considered as in ceaseless motion alternating in equilibrium opposite directions where they form vortices or sinusoidal waves. At some sites, these one dimensional waves are associated with their one dimensional cognates in a plane (or two dimensions) and this plane can hinge in values from the collinear of the parent to the orthogonal of the pair. There is thus dimensional variety.
4. In received knowledge, these features apply with considerable theory and experiment to waves from the very small wavelengths (cosmic or X Rays) to those at radio wavelengths in other words to the electromagnetic parts of the spectrum.
5. There are reasons to believe that the structure of space waves in this part of the spectrum (transverse waves) may apply to proposed space waves of lower wavelengths from supersonic and sound to elf waves (linear waves) as proposed anew in this article.
6. Special features apply to the dynamism accompanying the dimensional transitions of transverse waves where for reasons with a medical or health impact, the mensuration of space waves hithertofore immeasurable, becomes significant in a way to be discussed.
7. Because the living system makes use of both transverse and linear waves and because from 6. the mensuration of longitudinal waves when dealing with the living system is as essential as is that dealing with transverse waves at their origin, it is important to construct an analogy at the level of space structure and function between the two wave types where no such analogy exists at the moment.
8. It is intriguing that the commonality between the two rests on a basis (at present theoretical only) of their dimensional differences. One of the more important values for this bridge may be in an appropriate math form (in logic) which at the same time is equally appropriate for handling the abstract requirements of space function in the form of set theory and fractals. All of these receive their basis in Boolean algebra. We could say then that a method for slicing the structure of a space interactive flux in the nature of a computer assisted tomography would be most valuable where the details of the scan can be made observable in auditory or visual modes as we presently discuss.

### Events at the Fermi Surface

The preceding discussion on the details of prolific events at the transition, space-real is capable of truncation at certain points notwithstanding that some of these points could be considered speculative.

Two key constants in the transition formalism are due to Planck and to Boltzmann. In any table of constants, they both use an energy form conveniently heat (as joules) qualified in the former by time and in the latter by degrees of temperature at the Kelvin scale. This is perhaps not surprising because both derived from the interrelations of waves with the heat part of the spectrum in the earliest treatment of thermodynamics.

The burden assumed in the previous discussion was the further discretization of energy, wherein space itself was one of the compartments so cleaved off, with its imaginary to or non-real status, not in question. The premise of temperature in two key constants is real enough and so introduces a paradox, more perplexing when as was discussed, both Einstein and Planck were eventually able to rid the formalism of the T term. In other words, there was a pure space compartment aside from that somehow and inextricably linked to temperature. Intriguing was the proposition that space may have two properties one directly linked with temperature in an unknown fashion, the other related to pure space structure and function without any real association.

As we have mentioned, Einstein reasoned nearly a century ago from his formalism, that the only possibility for an energy devoid of Boltzmann's temperature, was to reside half of the zeropoint energy in the Maxwell radiation field but the other half resided in pure space itself. This leads to the interesting possibility that coherence of the pure space zeropoint energy from any cause could itself execute a radiation and just such a proposal was made some years ago as we mentioned by Davies and by Onruh. The interesting history of the idea has seen the radiation causal event considerably diminished from the original Davies proposal so that now minute space fabric tears such as in a collapsing bubble of ultrasonic origin have been suggested as the origin of light flashes accompanying the collapse. We note that the event would propel a greatly enhanced velocity even supriluminal in magnitude as compared to classical radiation speed.

Perhaps the most important value of this assignment of the radiation from a tear in space is that its waves are eventually paralleled in their origin to a parametric event such as in non-linear optics where the wave generation is always time symmetric. Time symmetry as discussed elsewhere, confers on the space wave, a non-cancelled non linear property associated with the exhibition of the Heinsenberg uncertainty in which resides an indeterminate and unknowable behavioural caprice which lies at the very heart of creativity. As Wunderman shows this circumstance is attached to the first eight to ten waves following the start (some use the word focus) of the fluctuation. This means that, to take advantage of the creative property of the wave, any system is most optimal where many start events are concentrated. A reasonable proposal would be that the biosystem is one with its creativity, which is advantaged by this primacy.

Just such events can be forecast in the renowned abrupt frequency and vectorial changes of phonons within the atom or atom complex within the first Brouillon or Jones zones respectively, as they show non-specular reflection from these boundaries. The possibilities for these abruptions toward parametric wave generation are considerable meaning that the zeropoint energy space elements with which the phonons are bathed, can generate the kind of radiation born of numerous starts at these sites. Indeed these starts and the several cycles to which they give rise would be proper candidates for two diverse purposes.

The first would be a return to the zeropoint energy to complete a cycle back to the atom itself, thus equilibrating the atom's nucleus-orbital electron energy cycling in relation to its stability.

The second could be a loop from this return available to a local growth point or points endowed with the same multistart property.

It is considered that the wave bundles or modes concerned would need be to privileged by some means to avoid their too ready envelopment by electric and magnetic fields to produce the classical transverse waves of electromagnetic radiation.

### Stability Considerations for ‘Frequent-Start’ Energy Generated at the Fermi Level

The value of frequent-start energy with its preservation of novelty derived from the energy of Planck's constant (Wunderman) to the biosystem would reside in its insulation from too-ready envelopment in the classical vestments of radiation due to electromagnetic and heat fields (FIG. 4). The following three diverse agencies might provide such protection to enable the juvenility of such a system to be permanently available such as might be of advantage to the creativity of the biosystem.
1. Heat itself where its density is kept to a minimum may be using its longer wavelength to prevent coupling of states generated at nano-meter wavelengths of the chemicals. The fraction kT may have such an insulating or protective function of space element states at the intra atomic and molecular levels.
2. Emissive radiation classically follows the exhibition of energy to the Fermi level space elements which results in such emission generated from singlet or triplet states both of which generations are subject to classical vestment as discussed. The triplet states fate however, is relatively long lived and its force can be preserved by linkage with a chemical whose valency electron spins are coherent such as sulphur and oxygen. Of these two elements the pull of the coherent spin is much the stronger toward the electron field in the kalabolic processes. (4) Once oxygen appeared in the atmosphere, great diversity of biosystem activity followed. Special systems for its widespread use in the organism soon (in evolutionary terms) evolved at the chemical level. Aerobic glycolysis soon superceded its sulphur-based anaerobic relative as an energy source.
3. An important agent in the biosystem adaptation to oxygen may have been a nervous system based on neurons and their connectives, the neuroglia. The cytomorphology of this strange shape is informative. More especially is its asymmetry of length-bregadth structure, measurable from microns (of most cells) to metres. Applying an ammeter to the membrane of the neurone quickly reveals a classical electromagnetic voltage but in quantum field theory terms this is not to say that the application of the electrodes merely made patent that which was latent. Expressed in the vein of this article, the act of measurement collapsed the waveform of space elements to the real state.

In this case the nervous system sufficiently steeped in oxygen becomes a “frequent-start” space energy source for distribution to the various tissues of the organism as they use this energy type in the recognised practice of making real.

The discussion this far has provided sufficient evidence to suggest that measurement, of first the availability of space elements at the Fermi surface and secondly their relation to classical radiative phenomena at that site, would represent a measurement of prime value to discovery of biosystem function.

### Review of the Push Pull Phenomenon to Incorporate Dimensionality

A further interpretation of the energy relationship of push pull and their influence on the cusp health and disease is a consideration of the energy subsumed between the two vectors of push (or stress) and pull (or tension) which authors suggest is primary in holding the whole biosystem together. Accordingly their imbalance from causes already described in terms of flow disturbance become significant and means to measure the imbalance at push and pull sites have been entered.

The two forces can be regarded as vectorial, meeting at a vertex and coming to circumscribe spaces in association with like force pains distributed over the area. These spaces can be seen as reflecting the average imbalance or disparity between the forces in either vector precisely, the measure required for quantitating the health status. Mathematically, precise expression can be given to this average of the dual forces in the form of the displacement of the space covered from a flat two dimensional plane. The effect is expectedly a curve in the plane which is measurable as a fraction of the integer dimensions known as a fractal. Fractal determination of space properties at the locus are thus key representatives of the average perterbation of the push pull dual.

### The Use of the Compact Disk to Probe Real and Space Wave Mechanics

### Introduction

We have referred to the problems associated with mensuration of a medium, space, where elements are not available to any sensorium of modern man or his instruments and which, in addition, suffer from an indeterminism or uncertainty from their very structure. At the same time we have emphasised historical steps that have occurred in the sequestration of the space element by way of ensuring its firm basis in the physical literature, steps going back slowly but inexorably for over one hundred years. The case for a more detailed probing of space seems worthy, more especially if important phenomena in say, living systems, can be assigned to this compartment. One such problem is the unavailability of a datum in such an insecure or ‘fluffy’ environ, a problem more aptly posed by certain religions which refer to ‘clapping with one hand’ or more basically, the carpenter attempting use of a hand saw without support for the timber. It has been written, that realisation of a form of space known as the aether in vogue some century or so ago and afforded almost universal discredit, suffered from an inability for measurement in that no datum was seen to be available.

The datum referred to is the compact disc which does provide continuing motion, does provide a datum in the form of its ridges (or ‘lands’) and is provided with a light source for diffraction-style interrogation of information from the datum lands which, as we have discussed, mandatorily contain the frequencies no matter of transverse or linear origin. It will be convenient to develop a modus is operandi for this machine to show how it simply and concurrently can incorporate in addition many of the features required to manipulate the apparent intransigence of space elements in their own ceaseless apparently random disarray.

We start synoptically with the behaviour of light imaged at edges of slits made in an opaque screen. The beam so produced has the light from the source mapping the slit but this light is interrupted by black bands or lines. The sequence across the light is thus hands of light and bands of no-light. The situation is capable of refinement if the slit is flooded with lens-collimated light applied at 90° to the slit. If the slit is observed with a telescope, the observer finds that the dark lines exist at precise rotations of the beam viewing angle where the light has been returned to the source alternating with light from the source not so obscured. It is found in the case of white light, that the bands appearing in the rotation correspond with precise frequencies generated in the source as evidenced by their colour. The conclusion we draw of interest to this interpretation is that a frequency band is discretised by an edge be this a hole or a grating provided the angle of the incident beam is fixed and that the bands so produced are light reflective toward the source so that the band is black. The fate of the transmitted light between the bands is undoubtedly complex in optical theory, but for present purpose, it can be regarded as dissipating the edge as space, which space contains no reflective agent and thus no electromagnetic radiation by which the beam is interrupted. Our subsequent discussions exploit these clear cut differences in the description of diffraction of light as now used in a more complex arrangement.

Fundamental Energy—Matter Relationships using the Compact Disk

In the theoretical discussion the behaviour of light at slits to produce the phenomena of diffraction was entered. This can be further refined, as mentioned, of what might be happening to involve space function in a more elaborate diffraction process. By the use of optical devices we can study the surface of a target invoking space elements thought to produce the frequency dissection noted at the edge of the slit not of electromagnetic radiation origin and now termed plasmons. As with the simpler wave splitting (termed diffraction) use is made of a light source whose beam axis is accurately rotated to produce a total internal reflection at a critical angle to the beam axis. At a point in the rotation, this time at a different critical angle, which angle avoids surface specular reflection and thus avoids reflection from an electromagnetic radiation itself the reflection will give way to emission of light corresponding precisely to a frequency but on this occasion the frequency is of a space element or elements in the plasmon which now becomes the intratomic or intramolecular space content referred to, adjoining the Fermi Surface in the preceding discussion.

The sequestration of internal from external reflection is important for the discussion if future consideration by physicists establishes the basic difference between reflection from electromagnetic radiation which is specular and that from space devoid of radiation (termed plasmons) which is now non-specular, We have made a case of several recent papers for a topologically side by side existence of radiation and non-radiative states of the cell extending to the tissue composed of the cell. Where the former is labelled by infrared radiation exhibited to a cell or tissue slice the adnexure of hot (radiative) and cold (non-radiative) zones in cell and tissue is clearcut as can be seen from the provision of adjoining slides prepared either for infrared only or histological stain only viewing. The former zones are extensive sometimes occupying over 50% of the tissue planes where their presence is adjudged as sufficient to be discerned by an appropriate scan marking the reflection status of say a laser beam.

The reasons for this clean cut division of cell or tissue in terms of thermal (electromagnetic radiative) or athermal (non radiative origin) underline our present ignorance of this surprising schism matched only by the importance of a measurement of the ratio of either state given the significance we have attached to the non radiative state as a capricious catalyst to the chemical which it dissipates and the central place of this property to the functioning biosystem.

An approach to this ignorance may reside in the different dimensional behaviour of two dissipative modes of imaginary energy discussed previously wherein the dissipation uses one and two dimensional paths. In the latter the requirements for internal reflection are met because the orthogonal mode projection is accompanied by an increase in refractive index. This means that the measurement of non-radiative energy in a biosystem component such as saliva, blood drop, urine drop or hair or other non invasive sample can be used to monitor its state including its balance state with radiative energy. As the laser is traversing the drop concerned their specific balance state will be measurable. Part of the value of the compact disc comes about from the operation of four contained principles. First it can interrogate waveforms derived from the functioning biosystem transforming the analogue into the digital state. There is no contra evidence that this de facto wave particle duality is not in considerable use in biosystem function so that the disc-associated software may be an appropriate place to reveal this alternation as valuable for the catalyst function on chemical valency. The disc was introduced with an emphasis on data compaction which is purely a biosystem property. Secondly the epitome of space elements as components equally surely means dealing with extradimensional measurement wherein benchmarks such as dimensional variation introduced by say lines of collected particles available in coloured noise makes for a grid to which the distribution of these space particles can be referred in the form of their fractals. It is not impossible that biosystems with their robust energy dissipation through Fermi surfaces or their equivalent in the non-metals, sulfur rises and falls in chemical potentials (or quantum numbers) of their component atoms as part of their embryonic foetal and adult development. The range of defective values of biomatter reported from our laboratories give a clue as to a likely dynamic variability of the imaginary or space energy packing of various tissues which, in turn, may be diagnostic of their origin in the organism in a non-invasive drop or sample mentioned previously. The compact disc associated software can handle these important parameters.

Thirdly a vectorial property is fundamental to the behaviour of the space energy dissipating the atomic orbital energy that imposes a balance on the particle progression through the Fermi surface wherein the atomic and molecular flow can suffer flow irregularity such as complex inversions which imposes a positive pressure or compression on the inflow side coupled to a negative pressure or tension on the outflow side. The balance of these two space zones is viewed as critical to the biosystem behaviour in its production of physiological progressing to pathological features. From theoretical discussions of what the laser beam sees of those two states in the disc movement we note that, over time, the two states are separable dependent on the inclination of the laser beam with reference to the orthogonal (or specular) status. It will be possible to visualize both the occurrence of the pressure alternation between plus and minus and its fate with time by suitable arrangements in the digital sensors in the associated software.

Lastly we refer back to discussions on the time symmetry property of the space element behaviour in dynamic mode. The wave progression is ever sensitive to the passage of time in marked contrast to the wave encapsulated in electromagnetic propagation where the flow of vectors ensures time cancellation in the property of time asymmetry. This means that if the energy component we demark in all these studies has' the clean cut schism from its radiative component, then the modification of time during the measurement process must always result in a better differential of the non-radiative component. In sum, compact disc measurement not only makes the non-radiative component measurable, it does so in ways which have a rational basis. These advantages are conveniently summarized by a figured comparison of a standard spectrum analyzer with the same spectrum now subject to an improved time change (FIG. 6d).

The disc operation from the manufacturer allows for the accidental application of injury, or other contaminant to the disc, by the process of “error correction”. Here the monitor applies the refracted signal iterated so many times so producing an extra elapse of time for disc rotation, such time becoming a measure of the “error”. The signal so provided is synthesised in software using an algorithm at a rate approaching the resolution of the binary bits. Where the error is of magnitude beyond the software to remedy, the output becomes modified by fragmentation of the signal and other discontinuities now to be described.

### Application of Living and Non Living Systems to the Disc Surface

As discussed previously, the information sought is ultimately the state of balance of the intra-atomic energy of the biochemicals involved. In equilibrium, it is proposed, neither the push nor pull aspects of imaginary pressure build up to arbitrarily excess and the metabolic (or growth) energy resulting is in arbitrary balance. Such a balance may, in our theories, and in terms of the chemicals involved in the dissipation, have been ensured by the passage of evolutionary time. We could imagine that small variations in push pull magnitude constitute the physiological state where energy of both radiative or non-radiative origin is added to the dissipation site in the atom. The list would include the addition of electromagnetic energy as in sunlight in plants or the results of nerve impulse additions or modifications of neuronal energy from endocrine or other origin. In animals enzymes such as proteases, kinases and so forth will be potent trausducers along space energy paths.

Now where the pressure builds up possibly from the introduction of foreign or unusual chemicals to the metabolism, coal tars, nicotine tars, viruses and so forth, the whole programme at the Fermi Surface is perturbed and an enhanced state, variously termed chemical potential, quantum number, dielectric value or sometimes “potentisation” is installed (FIG. 2c). The imbalance has now trended toward pathology.

Potentising is used for the build up of imaginary pressure in serially diluted water as practised in the preparation of homeopathic remedies. The principles involved may be important in the access of dimensional changes mentioned previously. Scholars of the process have thus determined at least three parameters for a rise in potency. These are:
1. Gravitation (seen as the space flux closely related to matter (or mass) presence and related relativistically to the space energy bending.
2. The circularity of the vessels used on the grounds that most of the effect is occurring at the wall surfaces. Square vessels perform poorly in allowing potentising. The circularity of blood vessel cross section may be important in potency stepwise increases between tissues.
3. The ambient magnetic field possibly because as a limited component (compared with the omnipresent E field) of the Maxwell equation field pair, the envelopment process enhances the dynamic mixing of the space components on their way to integration with the Maxwell fields.

### Knots

In disturbed metabolic equilibrium of the sort nominated, it is often intimated that the vortices concerned have rotated themselves into knots (FIG. 2c). It is equally intimated that relief of the knot with contingent restoration of the space flow should represent a most rational attempt at relieving any pathology associated with the impediment. Were this knot to show up as a between—set dimensions value and, were we to multiply the space elements, now manipulated as pieces, to form a known element dilution, it may be possible to define the dimensionality of the knot. Substitution of a known simpler topological arrangement of fractals for that site could metaphorically “undo the knot” (FIG. 2b). It is reasoned that the build up of spurious or parasitic space elements may then subside, assuming that the push pull equilibrium to be already tabulated or available a priore for that site in dimensional terms, can be restored. This would constitute a rational therapy sanctioned and limited only by the formalism used in its synthesis. The response could be supraluminal in velocity.

### Use

We assume that a CD machine of maximum variability of its parts and its operation is available so that for the various parameters, speeds, grids, gate filters and so forth, to be met for the measurements intended, there will be appropriate calibrative possibility.

The following experiments are suggested:
1. To determine the influence of ambient laboratory space on intratomic and para sound wave space or the reactive space elements, to the zeropoint energy fluctuations for that system. These are the phonons. A first approach will be the use of the disc in a Faraday cage or similar electromagnetic radiation shield.
2. Experiments to run the disc with congruent pen trace records of ambient pressure and temperature.
3. Conducting the playback in “mu” metal or other magnetic field shielding material cover. From related electronic field states, the playback to be related to spark discharges to simulate the E field.
4. Conducting the playback beneath a photodiode screen to relate the gate filter effect to radiation effects as Popp does with his ‘biophotons’.
5. Conducting the playback inside a cell where the refractive index can be varied such as in moats of sugar solutions, likewise DNA in salt solution, in a surrounding sleeve would be expected to affect plasmon response as in the “phantom” DNA effect in spectrophotometry.
6. Conducting the playback in a circular versus a pyramidal or cubic housing of aluminium foil.
7. Conducting the playback in the presence of a 10 kg block of lead and in the presence of the same mass as lead sheet say 3 mm thick.
8. Comparing results of the same gate filters in night versus day conditions as well as over seasonal conditions and solar and lunar phases.
9. Conducting the experiments in gas-sealed chamber using (a) air, (b) nitrogen (c) acetylene as experimental alternatives.

Given the opinions of two key investigators of the potentising process, gravitons play a major part in the space element equilibrium. Under those circumstances, trivial though gravity fields over such short distances may measure, the inverse square law may apply so that the above experimental arrangements may have negligible effects over magnitudes represented in the propinquity of the barrier filter.

### General Conclusions

### A New Acronym CAFST

1. The generality of the transition, imaginary to real is so widespread in the physics literature of the last century since the advent of knowledge of the quantum field, that any analysis of its process must have widespread application. In this report, the penchant has been toward that most complex of systems, those sustaining the life process but its meaning will apply equally to a requirement of any use of image or sound analysis.
2. The essence of the life system as with the climate system lies in its incessant movement in the performance of process with requirement for an analysis of the generality of the transition mentioned in 1. The rotation of the analyser in the case of the compact disc places the disc in a unique position and possibly this position has not been considered manipulable as an interrogator before. With a basis in analog-digital transition, in compressed storage of large volumes of information, with its essence in continuing movement and with an inbuilt provision for dimensional analysis, the compact disc is little short of a complete analog for the functioning biosystem. Its structural and functional attributes so parallel the living system that its invention cannot fail but to have included many detailed aspects of what is ‘out there’ in the best of intuitive hunches of past inventions.
3. An analysis of space elements provides a pleasing fit with emergent ideas on the universality of cosmic structure and function and of the place of earthbound living organisms in this hierarchy.
4. By insight of these same elements, the nature of the creative process starts to emerge and, to be part of it, gives satisfaction at least to the scientist and to the artist alike. An incipient elation is derivable from the experiments.
5. The analysis of space events at the Fermi Surface of atoms where two dimensions meet is productive of this creativity in the radiative energy and its reality which succeeds them. Such an analysis coupled with its interrogation on a rotating surface has the effect, in time, of a layered scan so that the process could well be termed a computer assisted Fermi Surface Tomography or CAFST. This must surely place the technique as a favoured servant to the Holy Grail of physics: the theory of everything.

### Legends to Figures

FIG. 5 comprises sketches of the state of waves proposed and described earlier in this specification:
a) Transverse waves seen in end section showing distribution of enveloped waves interspersed with non-enveloped waves, the former real the latter space or imaginary.
b) Lateral view of the same distribution in their respective enveloped sine wave and vorticeal states. An edge of matter is shown on the left of the diagram. For discussion purposes the vorticeal streets can be termed plasmons.
c) The longitudinal state of the imaginary sound wave is sketched here with islands of compression shown thickened. An imaginary plane intersects one of the compressions. The space elements of this plan are thus orthogonal to the longitudinal wave. These latter are termed ridgelets.
d) The analogous state of waves with a frequency from radio to cosmic. Orthogonal waves intersecting the collinear waves are shown with attached letter c leading putatively to carriage of pelagic matter eg electrons in opposed pairs such as in the superconductive state. The collinear waves are paired with motion either way. (arrows)
e) As for (d) but the orthogonal are shown enmeshed in the electron-nuclear energy systems of the atom. For convenience waves of either axis are shown as simple sine waves.
f) A modified picture of the Wunderman plan where the numbered integers of the axial (Euclidian) elements are intersected by orthogonal planar “flags” in various states of furling of their quartiles to emphasise his notions of the structure of Planck's constant h. The indeterminancy or unknowability of the furling state lies at the heart of the non-linearity of such waves. This contrasts with coincidence of the same wave with periodicity of time which is faithful to the arbitrary time intervals shown.

### FIG. 6

a) A sketch of the atomic nucleus and its orbitals as in FIG. 5e) to show the collinear wave distributing orthogonals (now shown as vortices) amidst the orbitals. The collinears provide a frame work for the latter (not shown) only to rejoin outside the orbital cloud and continue their collinear flight. The various vortices better illustrate a property of energy push-pull in the Fermi layer amongst the orbitals.
b) A sketch of a small section of the Fermi surface. The orthogonal vortices of figure Ga) are now shown as classical sine waves of varying frequency passing through the orbital energy cloud of one or more atoms. The balanced arrows represent the two way space energy flight through the orbital cloud with an idealized equilibrium flow to extra atomic space on either side. At bottom is a time scale with arbitrary divisions.
c) As for FIG. 6b) where the equilibrium flow is now considerably disturbed. The left side continues the pumping to be expected of zitterbewegung but the right side is imbalanced so that pressure in the Fermi Surface Elements is now raised. The arbitrary time scale shows that were timing available for the event, the pressure rise would be sustained in its measurement (see earlier description). To the left of centre the various flight paths are aggregated into what is in anthropomorphic terms a knot in contrast to the unknotted condition in FIG. 6b).
d) Lines representing spectral lines for an arbitrary compound in the living state such as in a laser Raman spectroscopy picture of a whole living microbe. The same component whose lines are now distorted by conducting the spectroscopy where, in this case, light beams and their target are in rotation.

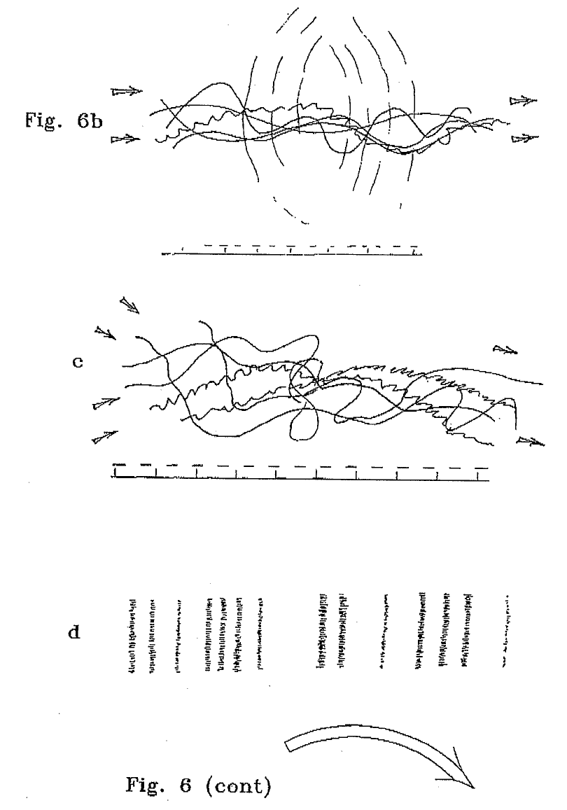

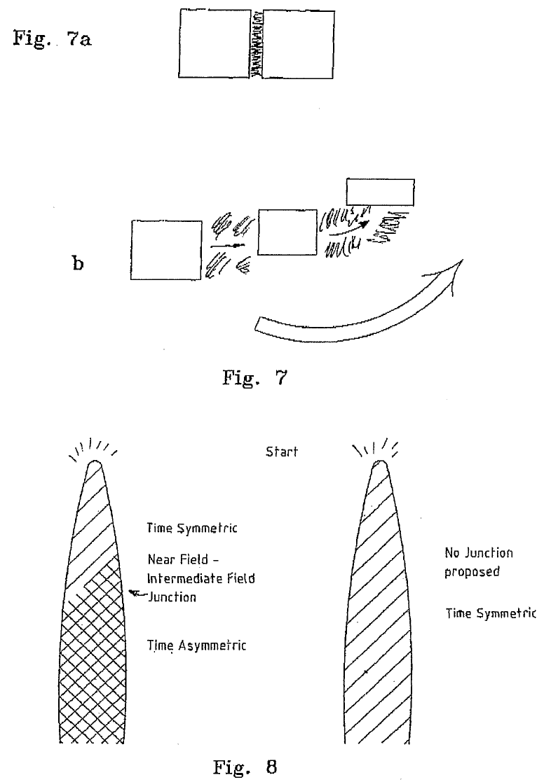

FIG. 7 Sketches of optical or visible frequencies on a compact disc:
a) Two pixels are shown adjoining a space element strip with vorticeal content. The frames are from a stationary picture.
b) Three pixels are shown, the left sided member as in FIG. 7a), the right hand pixel now turned orthogonal to the axis of the left hand. The frames are from a rotating picture where most of the intraatomic space lies between the pixels where its time symmetric state (as opposed to the time asymmetric state see FIG. 8) allows a slurring of the space elements which follow time involved in the rotation. The orthogonal elements have determined that the transposed energy state with its original site elements is displaced (in the sense that Clerk Maxwell used this term) to the site of the new image

FIG. 8 Modification of a sketch to accommodate the importance of “start” point or focus of a fluctuation as its nascency from space and its impact on the ability to measure such a state on rotating media. In the left hand figure, the hatching over the first eight-ten wave lengths from the start (Wunderman) indicates that time is synchronous with the wave generation behaviour whereas in subsequent wave lengths of its life history, most especially at the near-field intermediate-field junction the wave becomes real from the event of its enclosure in electric and magnetic fields of Maxwell. The progress becomes time asymmetric. In the right hand figure the particular fluctuation has become privileged in avoiding the enclosure. Measurement on a rotating disc permits discrimination of these two states, left and right. This is significant in the belief that the life history which includes the possibility of a system fully availing itself of time symmetry properties, may be important in the bio-system.

The above describes only some embodiments of the present invention and modifications, obvious to those skilled in the art, can be made thereto without departing from the scope and spirit of the present invention.

1.-109. (canceled)

110. A method for analysing function of a biosystem based on analysis of a sample taken from a portion of said biosystem, said method comprising:
exposing said sample to incident energy derived from an energy source;
receiving radiated energy from said sample consequent to impingement of said incident energy on said sample;
passing at least a portion of said radiated energy through a transducer thereby to derive an information signal which characterises an aspect of said sample;
and analysing said information signal to produce biosystem data which can be used to identify said aspect of said sample.

111. The method of claim 110 wherein said information signal includes a real component and an imaginary component.

112. The method of claim 111 wherein said imaginary component is used as a basis for characterising of said aspect of said sample.

113. The method of claim 110 wherein said aspect of said sample is a disease or malfunction.

114. The method of claim 110 wherein said aspect is used to characterise a disease or malfunction of an associated portion of said biosystem.

115. The method of claim 110 wherein said biosystem is a mammalian system.

116. The method of claim 115 wherein said mammalian system is the human body.

117. The method of claim 110 wherein said biosystem includes soil.

118. The method of claim 110 wherein said biosystem comprises an agricultural system.

119. The method of claim 110 wherein said step of analysing said information signal includes comparing said biosystem data derived from said sample with biosystem data derived from samples associated with a predetermined aspect of said biosystem.

120. The method of claim 110 wherein said aspect comprises a disease state.

121. The method of claim 110 wherein said aspect is characterised at the atomic level.

122. The method of claim 110 wherein said aspect is characterised with reference to the Fermi surface of atoms comprising said sample.

123. The method of claim 110 wherein background reference data is injected into said radiated energy.

124. The method of any preceding claim 110 wherein said sample is scanned repeatedly by said incident energy.

125. The method of claim 124 wherein said sample is placed on a platform which is rotated relative to said incident energy thereby to cause repeated passes of said sample through said incident energy.

126. The method of claim 110 wherein said incident energy derives from a laser source.

127. The method of any preceding claim 110 wherein said step of analysing said information signal to produce biosystem data is conducted in real time.

128. A device for analyzing biosystem function of a biosystem based at least partially on analysis of a sample taken from at least a portion of said biosystem, said device comprising:
a source of energy for exposing said sample to incident energy derived from said source of energy;
at least one sensor for receiving radiated energy from said sample consequent to impingement of said incident energy on said sample;
a transducer for receiving at least a portion of said radiated energy from said at least one sensor so as to derive an information signal which characterises an aspect of said sample;
a processor for receiving said information signal from said at least one sensor wherein said processor analyses said information signal to produce biosystem data which can be used to identify said aspect of said sample.

129. The device of claim 128 wherein said incident energy includes laser radiation.
